(19) United States
(12) Patent Application Publication
(10) Pub. No.: US 2017/0239032 A1
(43) Pub. Date: Aug. 24, 2017

(54) BIODEGRADABLE VASCULAR FILTER

(71) Applicant: Novate Medical Limited, Dublin (IE)

(72) Inventors: Stephen A. SOSNOWSKI, Vista, CA (US); David ROSENTHAL, Marrietta, GA (US)

(73) Assignee: Novate Medical Limited, Dublin (IE)

(21) Appl. No.: 15/279,742

(22) Filed: Sep. 29, 2016

Related U.S. Application Data
(63) Continuation of application No. 11/281,128, filed on Nov. 17, 2005, now abandoned.

(51) Int. Cl. A61F 2/01

(52) U.S. Cl. CPC A61F 2/01; A61F 2002/016; A61F 2230/0006; A61F 2230/0091; A61F 2250/0031; A61F 2210/0004

(57) ABSTRACT

Novel enhanced products and processes for trapping emboli utilize self-expanding skeletons and biodegradable polymer systems, for example stent-like Nitenol® elements and PLGA, to address longstanding issues related to thrombus capture without deleterious impacts on the vasculature or other negative artifacts of the procedure by at least partial post-use dissolution in situ.

### BACKGROUND OF THE DISCLOSURE

The present disclosure relates to novel enhanced surgical tools. In particular, the present disclosure describes apparatus useful for vascular surgical and interventional radiological procedures having improved trapping surface.

The documented needs, for example, for vena caval filters have driven the development of new devices to prevent travel of emboli during surgical interventions. Providing larger and more efficient trapping surfaces while minimizing insertion issues is a longstanding need in the art, and the advent of retrievable IVC filters merely underscores the need without addressing the issues which plague current treatment modes and modalities.

When surgical or radiological interventions are done, and when patients present with systems or disease etiologies that relate to the generation of blood clots, or thrombus, medical devices have been introduced which function to prevent these from passing into other areas of the body where they can be harmful or lethal.

Exemplary devices which have been used to manage such conditions have generated a plurality of longstanding needs yet to be addressed. Incorporated by reference herein, and illustrative of these predicate devices having generated most of these shortcomings are found in the following United States Letters Patents, which serve to define the state of the art prior to the advent of the instant teachings: U.S. Pat. Nos. 6,932,832; 6,669,721; 6,666,882; 6,652,558; 6,582,447; 6,669,721; 6,605,111; 6,517,559; and 6,267,776.

Each of these references has been studied, as have the devices that embody them, as discussed below, and found to be differentiated from the subject matter of the present invention. For that reason and because of the urgent needs to provide for treatments for patients that work better than the state of the art, the instant disclosure is hereby offered for consideration as an instantiation of progress in science and the useful arts, and Letters Patent hereby earnestly solicited for that reason and each of those set forth below and claimed.

### SUMMARY OF THE DISCLOSURE

The present inventors have overcome longstanding issues in preventing recurrent pulmonary embolism, among other things, by percutaneous placement of an improved biodegradable filter in the vena cava. This enhanced treatment modality addresses pulmonary thromboembolism when anticoagulants are contraindicated, treats thromboembolic disease, addresses massive pulmonary embolism and chronic, recurrent embolisms better than existing devices.

According to a feature of the disc a biodegradable vascular filter system, which comprises, in combination, a self-expanding apparatus which undergoes a phase change enabling it to move from a first, compacted position to a second, expanded position. operatively connected with a plurality of polymeric string-like members, which members expand from a slackened to a tensioned state in conjunction with the phase change of the associated apparatus, wherein the system when emplaced in at least one of a vessel and a lumen is effective for trapping thrombi traveling therethrough.

According to another feature of the disclosure a process for mitigating insult and injury by thrombus comprising, in combination, providing a vascular filter device further comprising a nitinol skeleton operatively linked to a biodegradable polymer, emplacing the vascular filter device at a desired location upstream of at least one of a surgical and an interventional procedure site, performing at least one of a surgical and an interventional procedure, and leaving the vascular filter device in situ.

Briefly stated, novel enhanced products and processes for trapping emboli utilize self-expanding skeletons and biodegradable polymer systems, for example stent-like Nitinol® elements and PLGA, to address longstanding issues related to thrombus capture without deleterious impacts on the vasculature or other negative artifacts of the procedure by at least partial post-use dissolution in situ.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a schematic showing a biodegradable filter according to embodiments of the present disclosure;

FIG. 2 is a schematic top view showing a biodegradable filter according to embodiments of the present disclosure;

FIG. 3 is a schematic partial perspective view of a biodegradable filter according to embodiment of the present disclosure;

FIG. 4 is a schematic side view of a biodegradable filter according to embodiments of the present disclosure;

FIG. 5 is a schematic side view according to embodiments of the present disclosure;

FIG. 6 is a partial plan view of a biodegradable filter according to embodiments of the present disclosure;

FIG. 7 is a side view of a biodegradable filter according to embodiments of the present disclosure; and

FIG. 8 is a side view of a biodegradable filter.

### DETAILED DESCRIPTION OF PREFERRED EMBODIMENTS OF THE DISCLOSURE

The present inventors have discovered a novel enhanced process and products to mitigate thrombic insult, injury and related and attendant harms. By combining a shape memory alloy, for example Nitinol®, and biodegradable polymer systems an improved surgical filter effective to arrest transmissions of thrombus is disclosed. Processes using various embodiments are also taught.

Those skilled in the art readily understand that a biodegradable polymer system includes any related biocompatible set of moieties approved, or to be approved, for use in animals. By way of illustrative example, poly(lactic-co-glycolic) acid (hereafter “PLGA”), is readily substitutable for any number of biodegradable polymers having a strong history of usage in U.S. Food and Drug Administration (“FDA”) approved devices.

Likewise, delivery systems are conventional, and used by all of the major cardiovascular disease companies, which must be given consideration in the design and execution of such medical devices. The trend in these devices is procurement of larger and more effective trapping surfaces and smaller and less invasive insertion systems.

Prominent examples of the other devices in these fields include the LP brand of filter from B. Braun, the Gunther Tulip™ brand of Vena Cava filter, and the Cordis Optease brand of permanent vena cava filter, in addition to the Recovery brand of filter system offered by Bard Peripheral Vascular, a division of C.R. Bard Incorporated. Unacceptably high records of adverse events are associated with all of these devices. It may be synthesized by the co-polymerization of glycolide and lactide. The present inventors have searched predicate devices and approaches but are unaware of other usages of PLGA such as those taught according to the present disclosure.

Likewise, although indications are clearly available for improved filters nothing which has effectively addressed and solved the problems at which the present invention is directed currently is known. By way of further example of the need for the present invention vena caval filters have commonly been adapted or used in a other lumens as needed.

Another known filter is the Greenfield brand of filter from Boston Scientific. Each of these devices have been studied and found to subject to various complications stemming from common challenges. The present disclosure overcomes such issues.

Filter occlusion, from trapped emboli, often results in adverse events ranging from renal failure, the need for heightened thrombolytic therapy, to death of the subject patients. Metal fatigue and fracture, poor flow characteristics and areas of stagnation also generate significant issues. Fixation hooks associated with known devices, and the high radial force associated with the deployment of known systems have also added vasculature insult and injury to the list.

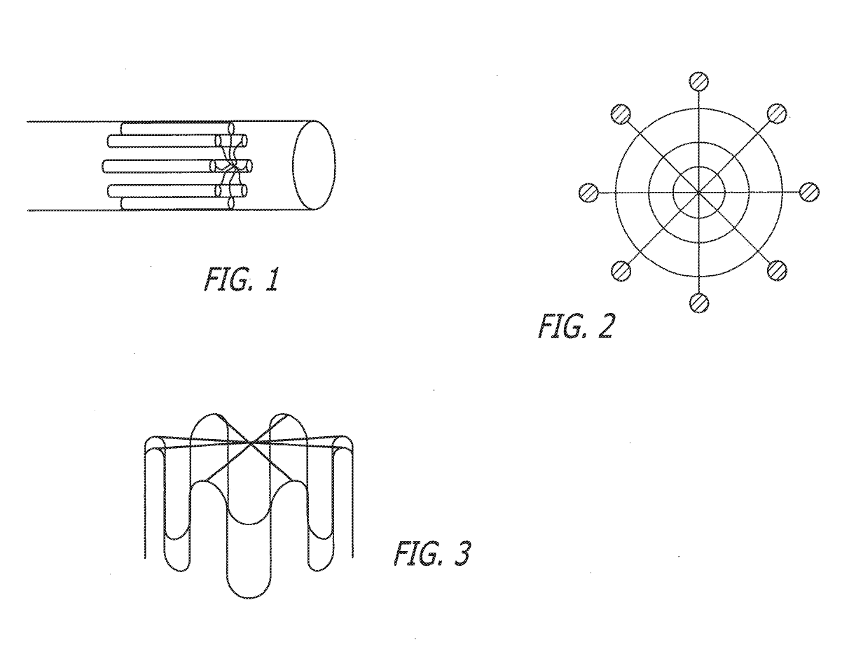

Turning now to FIG. 1, novel enhanced biodegradable filter is generally and schematically illustrated as device 101 struts 111, as deployed leverage off of the benefits of Nitinol®, or “spring steel” which moves from a first (compacted) position to a second (expanded) condition upon release within an environment having a higher temperature—such as the desired lumen of a vessel. Artisans readily understand restraints may delay this expansion as commonly practiced within the catheter arts. PLGA matrix 113 provides for a trapping mechanism when tensioned by the expansion of Nitinol® struts 111 as the ‘spring steel’ move from a first to a second position.

Referring now also to FIG. 2, it is shown now expanded struts 111 of device 101 can render PLGA (or any other biodegradable polymer system, as set forth and discussed above and claimed below) matrix 113 effective to trap emboli, without the constraint of concomitant flow restriction. Deployment of device 101 does not cause vessel damage through high radial force, nor do damages by ripping into the vessel wall. Rather Nitinol® struts 111 merge gently with vessel walls, growing into the neointima of the vessels.

Turning now to FIG. 3, a stent-like embodiment of device 100 features Nitinol® hoops 117, whose memory allows them to be situated within a delivery catheter and through minimally invasive techniques, delivered to an appropriate site.

The medical device usage of shape-memory alloys, whose function as is well know to those skilled in the art in accordance with the PALMAZ-type of self expanding stent (Cordis Endovascular, Johnson & Johnson), to render device 101 effective to be delivered by known systems of catheters, and to be placed at an appropriate juncture in a vessel without damaging the same. For example, placement in any known vessel by a femoral cut-down using a trocar, introducer and guidewire system (available from Medtronic AVE, Guidant, Edward LifeSciences LLC or Cook Endovascular as approved by the U.S. FDA), is conventional.

The benefits of stent-like device 101, with for example PLGA web 113, are significant in comparison to known teachings. For example, as opposed to leaving the filter in the patient, or attempting to retrieve the same by dragging it out, each of which does more harm than good—the instant disclosure teaches leaving the device in, and using the PLGA to dissolve which permits the remainder of the assembly to dissolve in situ.

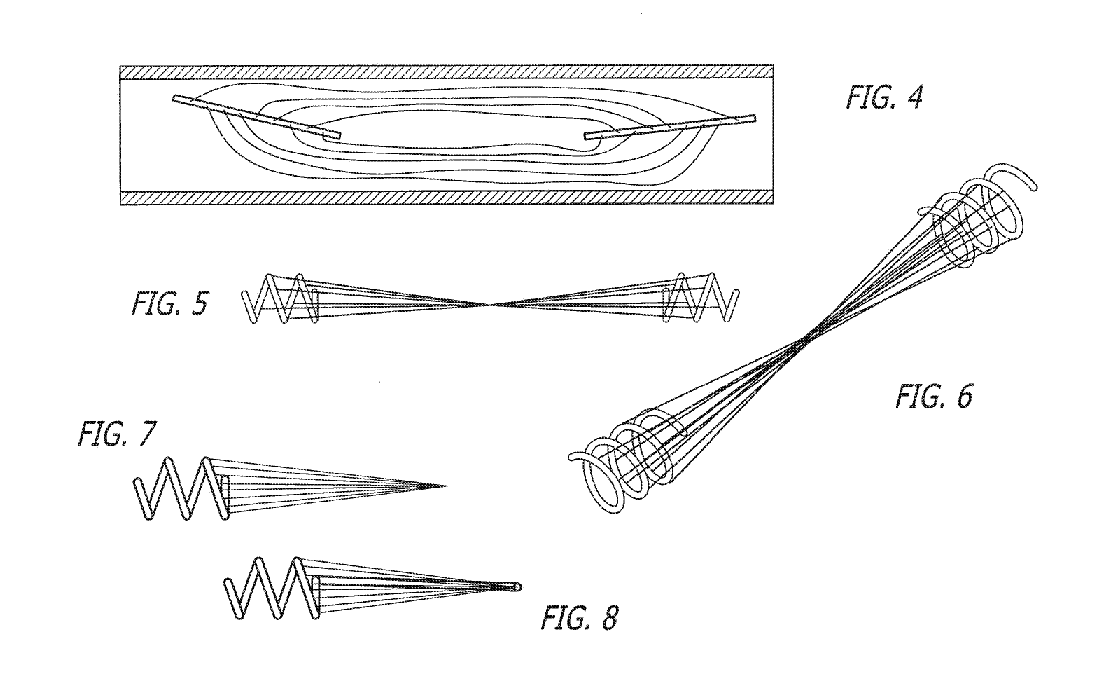

Turning now to FIG. 4, an alternate embodiment is shown which has closer analogy to the Cook Endovascular Bird-Nest® brand of device. This biodegradable filter 201, once again is comprised of Nitinol® struts 211, which are shown in a first (compacted) condition within catheter/delivery system 222. Once more, “spring-steel” may be chilled, cooled or otherwise restrained to maintain this first state. Nitinol® 211 undergoes a phase change from austenite to martenite upon a correct temperature change and the “memory” it has allows the health-care provider to size it appropriately for the desired vessel. PLGA is an effective polymer system, and those skilled in the art will understand that others may be used as well.

FIG. 5-FIG. 8 demonstrate a second or expanded state of Nitinol® struts, respectively proximate 311 and distal 312 (411, 511) as used to filter emboli in different vessels. FIG. 5, for example may be used for pregnant patients with thromboembolism, In such a disease state, extensive illofemoral deep vein thrombosis with thrombolytic therapy or surgical thrombus—generating procedures are a major area of concern.

The devices in the field are designed to trap emboli during these procedures, but generally add more risk factors than they prevent. The instant disclosure overcomes these issues and allows surgeons and interventionalists an option.

It is also prominent in the literature that permanent vena cava filters often cause pulmonary embolisms, and other significant complications many of which are addressed and overcome by the instant teachings.

FIGS. 6-8 show customized versions which may be used as temporary filters.

Likewise, FIG. 7 and FIG. 8 use Nitinol® stent-like members 411, 511 for trauma and orthopedic surgery with PLGA (and the like polymers) 413, 513 being custom-tailored also for pediatric, hepatic, biliary usage, Anchor 538 may also be used for smaller vessels or specialized approach where lumens are challenging to access or require alternate positioning means. Such usages are within the ambition of surgeons or interventional radiologists of skill in the art, and so further discussion is omitted at this time.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A biodegradable vascular filter system, which comprises, in combination:
a self-expanding apparatus which undergoes a phase change enabling it to move from a first, compacted position to a second, expanded position, operatively connected with;
a plurality of polymeric string-like members, which members expand from a slackened to a tensioned state in conjunction with the phase change of the associated apparatus;
wherein the system when emplaced in at least one of a vessel and a body lumen and is effective for trapping thrombi traveling therethrough.

2. The biodegradable vascular filter system of claim 1 the apparatus further comprising a plurality of strut members which are radially elongated in the first compacted position.

3. The biodegradable vascular filter system of claim 2, the apparatus further comprising an attachment between each of said polymeric string members and at least one of the plurality of strut members.

4. The biodegradable vascular filter system of claim 3, further comprising at least a supplemental restraining mechanism that maintain the self-expanding apparatus in the first position until a desired release time.

5. The biodegradable vascular filter system of claim 3, wherein the apparatus further comprises at least one shape memory alloy selected from the group consisting of Nitinol® and other biocompatible metals.

6. The biodegradable vascular filter system of claim 4, wherein the apparatus further comprises at least one polymer system selected from the group consisting of LGPA and other polymers which dissolve at a predetermined time.

7. The biodegradable vascular filter system of claim 3, wherein the apparatus further comprises Nitinol® struts and a plurality of string-like members comprised of LGPA.

8. A process for mitigating insult and injury by thrombus comprising, in combination:
providing a vascular filter device further comprising a shape memory alloy skeleton operatively linked to a biodegradable polymer;
emplacing the vascular filter device at a desired location upstream of at least one of a surgical and an interventional procedure site;
performing at least one of a surgical and an interventional procedure; and
leaving the vascular filter device in situ.

9. The process of claim 8, wherein the shape memory alloy is Nitinol® and the polymer system consists essentially of LGPA.

10. A method for treating at least one of pulmonary embolism and a disease state characterized by generation of thrombus, which comprises the steps of:
operatively disposing a filter in accordance with claim 7 within a desired vessel of a patient;
causing the filter to expand from a first compacted to a second expanded position;
capturing at least of emboli and thrombi for a desired period of time; and
having the web or matrix dissolve.

11. A method for treating of claim 10, wherein the vessel is the vena cava.

12. A method for treating of claim 10, wherein the vessel is the aorta.

13. A method for treating of claim 10, wherein the vessel is among the peripheral vasculature.
